(19) United States
(12) Patent Application Publication
(10) Pub. No.: US 2026/0345336 A1
(43) Pub. Date: Nov. 5, 2026

(54) METHOD FOR MAKING A PROSTHETIC IMPLANT AND PROSTHETIC IMPLANT SO OBTAINED

(71) Applicant: LIMACORPORATE S.P.A., San Daniele Del Friuli (IT)

(72) Inventors: Michele BULFONE, Tarcento (IT); Michele PRESSACCO, Martignacco (IT)

(21) Appl. No.: 19/487,642

(22) PCT Filed: May 24, 2024
(86) PCT No.: PCT/IT2024/050106

§ 371 Date: Nov. 25, 2025

(30) Foreign Application Priority Data
May 25, 2023 (IT) 102023000010665

(51) Int. Cl. A61F 2/30; A61F 2/34; A61F 2/40

(52) U.S. Cl. CPC A61F 2/3094; A61F 2/34; A61F 2/4081; A61F 2310/00023; A61F 2310/00029

(57) ABSTRACT

Method for producing a prosthetic implant, comprising the following steps:—providing a first component made of a first metal or metal alloy and equipped with a first perimeter part;—providing a second component made of a second metal or metal alloy and equipped with a second perimeter part;—creating an assembly formed by said first component and said second component, by means of a coupling by interference between said first perimeter part and said second perimeter part;—inserting said assembly into an isostatic press for a given period of time until diffusion bonding is obtained between said first component and said second component by means of hot isostatic pressing.

### FIELD OF THE INVENTION

The present invention concerns a method for producing a prosthetic implant and a prosthetic implant thus obtained. The prosthetic implant can be used, for example, to restore the hip or shoulder joint.

### BACKGROUND OF THE INVENTION

In the field of orthopedic prosthesis it is known to make orthopedic or prosthetic implants having a hemispherical cavity that acts as a positioning and rotation seating, for example in the case of a hip joint, for the head of a femoral prosthesis. The femoral head can be made of polyethylene, polyether ether ketone (PPEK), ceramic materials, or other.

The prosthetic implant can be made by means of two hemispherical metal components that are produced and coupled using various methods, for example by means of diffusion welding, known in the metallurgical sector as “diffusion bonding”.

This technique consists of making two metal surfaces adhere to each other through the action of the temperature and pressure, and is completed using mechanical elements or external apparatuses in direct contact with said components in order to press them against each other.

The direct contact with an external apparatus made of a different material can lead to plastic deformation of the surface of the components to be coupled, which have a certain porosity, to morphological modification of the reticular structure constructed and optimized to increase bone growth, and to possible chemical contamination or incorporation of foreign residual material that can reduce the osteoconduction of the porous surface.

Known methods for producing prosthetic implants by means of diffusion bonding therefore require the direct contact, at least along one axis, of an external mechanical apparatus or means to exert the right pressure on them, and this procedure can result in defects or unwanted morphological and structural variations of the final orthopedic implant. Known methods are also limited by the geometry of the components to be joined. For example, it is difficult for an external apparatus to apply a homogeneous pressure on hemispherical surfaces, typical of the components to be joined.

Known apparatuses and methods are also rather complex, so their implementation is often challenging in terms of costs and production times.

Document WO 01/54561 A2 describes a prosthetic knee joint having an articulation surface formed as a sintered polycrystalline diamond compact component. In particular, a sintered polycrystalline diamond compact component gives a chemical bond and a mechanical grip between the articulation surface and the material of a metal-containing, that is, metallic, substrate. The polycrystalline diamond compact component can be used for the femoral head and/or acetabular cup. The polycrystalline diamond compact component provides a chemical bond between the material of the metallic substrate and the diamond crystals. The method for producing the polycrystalline diamond compact component provides to sinter the diamond crystals together and on the metallic substrate at high pressure and high temperature. Generally, an amount of diamond raw material, supplied as diamond powder or crystals, is positioned adjacent to a metallic substrate prior to sintering. The interface between the diamond powder and the substrate material is a critical region where the bond of the diamond material to the metallic substrate has to occur. Once the diamond powder or crystals and the metallic substrate are assembled, the assembly is subjected to high pressure and high temperature in order to cause the bonding of the diamond crystals with the diamond crystals and with the substrate. The resulting structure of the sintered polycrystalline diamond material bonded to the metallic substrate defines the aforementioned polycrystalline diamond compact component, that is, a composite structure of two materials of different nature, that is, diamond crystals and metallic substrate.

The technique for loading the raw material used for the diamond is critical to the success of the final product design. The diamond raw material has to be loaded with a uniform density in order to produce a component free of unwanted distortions. To this end, there is provided a rather complex apparatus comprising a rotating rod with an end adapted to the sizes and shape of the piece to be produced. For example, if the part to be produced is a femoral head or an acetabular cup, the end of the rotating rod is hemispherical. A compression ring is provided having a hole through which the rotating rod can protrude. There is provided a die or mold with a cavity which is also adapted to the sizes and shape of the piece to be produced. To load the diamond raw material, the rotating rod is positioned in a drill chuck and the rotating rod is aligned with the center point of the die or mold. The mold is loaded with a known amount of diamond material. The rotating rod is then rotated around its longitudinal axis and lowered into the mold cavity to a predetermined depth. The rotating rod comes into contact with and rearranges the diamond raw material during this operation. The rotation of the rotating rod is then interrupted, and the latter is clamped in position. The compression ring is then lowered around the outside of the rotating rod to the point where the compression ring comes into contact with the diamond raw material in the cavity of the die or mold. The part of the compression ring that comes into contact with the diamond is annular. The compression ring is compressed up and down to compact the diamond. This type of compaction is used to distribute the diamond material throughout the cavity at the same density and can be performed in stages to avoid the formation of bridges.

To sinter the diamond raw material, assembled and loaded as described above, into polycrystalline diamond both heat and pressure are required. Heat is supplied electrically while the part is subjected to pressure in a press. A heater unit is used to supply the required heat. A refractory metal container containing the loaded and pre-compressed diamond raw material is inserted into a heater unit provided with a group of graphite heating tubes. Once the heating tubes are supplied with electrical energy, they generate the heat necessary for the formation of the polycrystalline diamond in the high-pressure/high-temperature pressing operation. Once prepared, the heater unit is inserted into a pressure unit for sintering in a high-pressure and high-temperature press. A cubic press or a tape press can be used for this purpose. The pressure unit is intended to receive the pressure from a press and transfer it to the diamond raw material so that the sintering of the diamond can occur under isostatic conditions. The pressure unit containing the refractory metal container inside which the diamond raw material is loaded and pre-compressed is then inserted into an appropriate press. Once the desired temperature has been reached by means of the heater unit, the pressure unit is subjected to the necessary pressure. The components of the pressure unit transmit pressure to the diamond raw material. Once the sintering cycle of the polycrystalline diamond compact components is completed, a cooling period is provided, then the pressure is removed. The polycrystalline diamond compact component is then removed for finishing.

The method described in WO 01/54561 A2, therefore, is not suitable to make a prosthetic implant by means of diffusion bonding of two hemispherical metal components since the nature and physical form of the materials described, that is, diamond powder or crystals and metallic substrate, is profoundly different, as well as the nature of the bond described in this document and the processes and mechanisms involved. Likewise, the machinery and apparatuses used in the method described in WO 01/54561 A2 are complex and laborious.

There is therefore the need to perfect a method for producing a prosthetic implant and a prosthetic implant thus obtained that can overcome at least one of the disadvantages of the state of the art.

In particular, one purpose of the present invention is to perfect a method for producing a prosthetic implant by means of diffusion bonding which allows to apply a homogeneous pressure on the components to be joined without using external apparatuses that exercise the required pressure on said components. This absence of direct contact, without any mechanical forcing, allows to eliminate any risk of plastic deformations or residual contamination of the generally trabecular structure of the prosthetic implant.

Another purpose of the present invention is to perfect a method for producing a prosthetic implant which allows to achieve an effective and uniform metallurgical bond by means of diffusion bonding even in the presence of components with partly hemispherical geometry, such as acetabular cups.

Another purpose of the present invention is to perfect a method for producing a prosthetic implant which allows to reduce the complexity and costs compared to what happens in the processes known in the sector.

Another purpose of the present invention is to make a substantially monolithic prosthetic implant, which has excellent properties of internal bone growth and biocompatibility in contact with the bone, and is resistant to wear when coupled for example to a femoral head made of materials such as polyethylene, PEEK, ceramic, or other.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes, a method according to the present invention for producing a prosthetic implant comprises the following steps:
providing a first component made of a first metal or metal alloy and equipped with a first perimeter part;
providing a second component made of a second metal or metal alloy and equipped with a second perimeter part;
creating an assembly formed by the first component and the second component, by means of a coupling by interference between the first perimeter part and the second perimeter part;
inserting the assembly into an isostatic press for a given period of time until diffusion bonding is obtained between the first component and the second component by means of hot isostatic pressing, therefore with specific pressure and temperature values.

Advantageously, this method allows to apply a homogeneous pressure on the components to be joined without using external apparatuses that exercise the required pressure on the components. This absence of direct contact, without any mechanical forcing, allows to eliminate any risk of plastic deformations or residual contamination of the generally trabecular structure of the prosthetic implant.

This method allows to obtain an effective and uniform metallurgical bond by means of diffusion bonding even in the presence of components with partly hemispherical geometry, such as acetabular cups.

Furthermore, this method allows to reduce the complexity and costs compared to what happens in the processes known in the sector.

According to another aspect of the invention, the first component comprises a concave seating with a hemispherical or at least partly hemispherical shape, and the second component comprises a cap with a hemispherical or at least partly hemispherical shape substantially mating with the shape of the concave seating.

According to another aspect of the invention, between the concave seating and the cap an interface is created with a substantially hemispherical or at least partly hemispherical shape along which the diffusion bonding is made.

According to another aspect of the invention, the coupling by interference occurs in a zone of contact between the lateral walls of the perimeter parts.

According to another aspect of the invention, the first component is made using a titanium-based alloy.

According to another aspect of the invention, the external surface of the first component has a porous or trabecular structure.

According to another aspect of the invention, the second component is made using a cobalt-based alloy.

According to another aspect of the invention, the first perimeter part and the second perimeter part are cylindrical or truncated conical.

According to another aspect of the invention, the first perimeter part provides a seating in which the second perimeter part, conformed substantially as a ring, is inserted.

According to another aspect of the invention, the coupling by interference between the components occurs through contact between the respective lateral walls of the first perimeter part and of the second perimeter part.

According to another aspect of the invention, the surface roughness of the concave seating and of the cap is about 1 μm Ra or less, preferably 0.2 μm Ra or less.

According to another aspect of the invention, the method provides a final finishing mechanical working step that follows the exit of the assembly from the isostatic press.

According to another aspect of the invention, the method provides a sintering step prior to the hot isostatic pressing step.

The invention also concerns a prosthetic implant comprising a first component made of a first metal or metal alloy and a second component made of a second metal or metal alloy, which are coupled by means of diffusion bonding obtained by means of hot isostatic pressing. The second component defines a concave seating or cup, in which the head of another insert or prosthetic implant, made for example of polyethylene, PEEK, ceramic, or other, can be inserted, during use.

This prosthetic implant is substantially monolithic, it has excellent properties of internal bone growth and biocompatibility in contact with the bone, and is resistant to wear when coupled to said head.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a lateral section view of a first component of a prosthetic implant according to the present invention;

FIG. 2 is a lateral section view of a second component of the prosthetic implant;

FIG. 3 is a lateral section view of the first and second components assembled and introduced into a hot isostatic press;

FIG. 4 is a lateral section view of the finished prosthetic implant;

FIG. 5 is a microscopic view of the structure of the prosthetic implant in the zone in which the components couple to each other.

We must clarify that the phraseology and terminology used in the present description, as well as the figures in the attached drawings also in relation as to how described, have the sole function of better illustrating and explaining the present invention, their purpose being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS

We will now refer in detail to the possible embodiments of the invention, of which one or more examples are shown in the attached drawings, by way of a non-limiting illustration. The phraseology and terminology used here is also for the purposes of providing non-limiting examples.

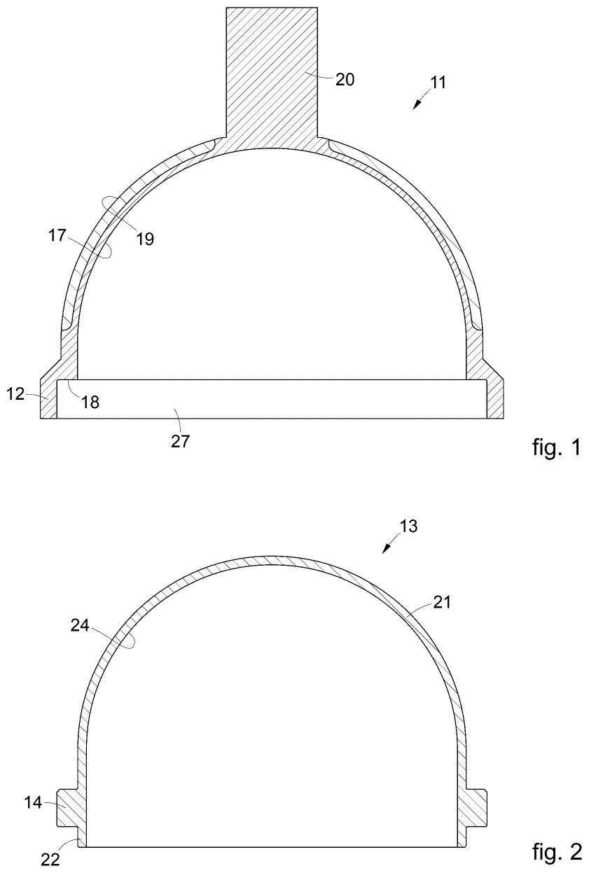

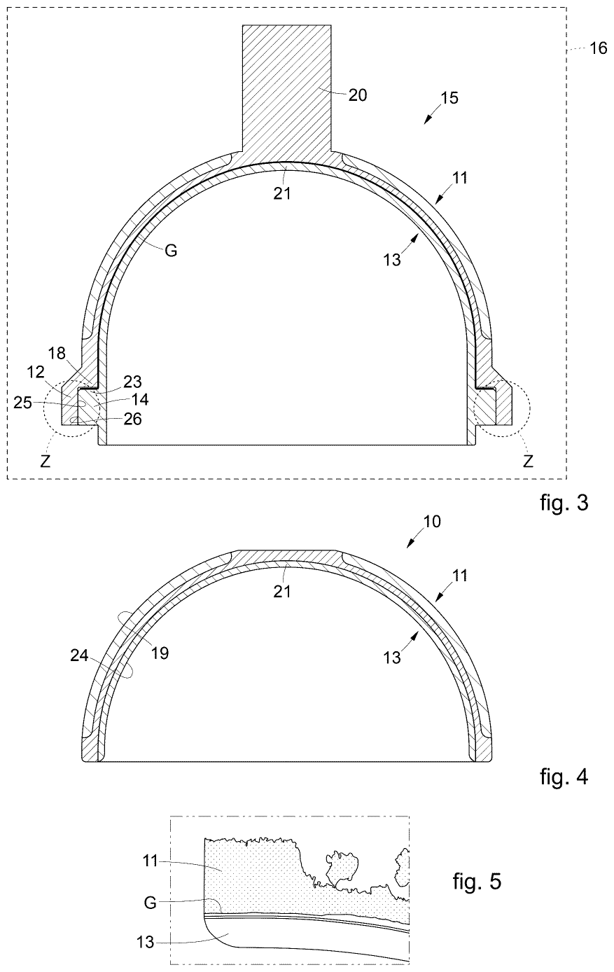

With reference to the attached drawings, a method for producing a prosthetic implant 10, shown in section view in FIG. 4, comprises the following steps:
providing a first component 11 made of a first metal or metal alloy and equipped with a first perimeter part 12, see FIG. 1;
providing a second component 13 made of a second metal or metal alloy and equipped with a second perimeter part 14, see FIG. 2;
creating an assembly 15 formed by the first component 11 and the second component 13 by means of a coupling by interference between the first perimeter part 12 and the second perimeter part 14, see FIG. 3;
inserting the assembly 15 into an isostatic press 16, see FIG. 3, for a given period of time until diffusion bonding is obtained between the first component 11 and the second component 13 by means of hot isostatic pressing, also known as HIP.

In the isostatic press 16, the pressure is applied by means of an inert gas, for example argon. The application occurs for a certain period of time at high temperature and pressure, for example between 50 MPa and 300 MPa and with temperatures ranging between 480 °C and 1300 °C. The internal environment of the isostatic press 16 can also be brought under vacuum.

As shown in FIG. 1, the first component 11 is provided with a concave seating 17 substantially with a hemispherical or at least partly hemispherical shape. The concave seating 17 is followed by the first perimeter part 12, which can be internally cylindrical, as shown, or truncated conical. Internally, the first perimeter part 12 has a seating 27 in which a step 18, in particular an annular step, is created. The first component 11 can be made using a titanium-based alloy.

The external surface 19 of the first component 11 can advantageously have a porous or trabecular structure, to promote osseointegration.

A shank 20 protruding from the external surface 19 allows to facilitate the movement and assembly of the first component 11 with the second component 13. The second component 13 is able to be inserted into the concave seating 17 of the first component 11.

In particular, the second component 13 comprises a cap 21 with a substantially hemispherical or at least partly hemispherical shape. The shape of the cap 21 is substantially mating with the shape of the concave seating 17.

The second perimeter part 14 of the second component 13 is substantially a ring that develops around the cap 21, in particular in proximity to a base 22 thereof. The second ring-shaped perimeter part 14 is then inserted into the first perimeter part 12, during the step of assembling the first component 11 and the second component 13.

The internal part of the cap 21 defines a concave seating 24, or cup, into which the head of another prosthetic element or implant can be inserted, during use. The second component 13 can be made using a cobalt-based alloy.

During the step of assembling the first component 11 and the second component 13, the second ring-shaped perimeter part 14 is inserted into the seating 27 of the first perimeter part 12, however the step 18 and the surface 23 of the perimeter part 14 which faces the step 18 are not in contact. See FIG. 3.

The coupling by interference between the components 11 and 13 therefore takes place between the respective lateral walls 25 and 26 of the first perimeter part 12 and of the second perimeter part 14. A substantially annular zone, or contact zone, Z of interference between the perimeter parts 12 and 14 is therefore defined. In other words, the coupling by interference takes place in a zone Z of contact between the lateral walls 25 and 26 of the perimeter parts 12 and 14.

Between the first component 11 and the second component 13, in particular between the concave seating 17 and the cap 21, an interface G is created with a substantially hemispherical or at least partly hemispherical shape with the absence of direct tight contact, and which allows to achieve a so-called “closed-porosity”, that is, defined as porosity not accessible to the inert gas provided in the isostatic press 16.

In particular, the contact zone Z realizes the aforementioned region of closed porosity.

According to some possible embodiments, the components 11 and 13 can initially have a zone of substantially punctual or in any case narrow contact in proximity of the corresponding apexes or poles.

The diffusion bonding occurs inside the isostatic press 16, where the assembly 15 is positioned for a certain period of time.

The isostatic press 16 has an inert internal environment with argon and vacuum, as a function of the phase of the thermal cycle applied. The thermal and pressure cycle can in fact be divided into different phases to achieve the complete diffusion bonding in the interface G. For example, different heating, holding and cooling phases can be provided in succession, variously combined and repeated. For example, a thermal cycle regulated by different holding temperatures and different cooling and heating ramp ups can be provided.

The hot isostatic pressing step can be preceded by a sintering step with specific temperatures and pressures. This step may be carried out inside the isostatic press 16.

The coupling by interference that allows to create a closed porosity between the two components 11 and 13 also exploits the differences in thermal expansion between the two different metal materials which, during a first phase of the thermal cycle, allows to increase the forces in the zone Z of contact between the perimeter parts 12 and 14, in particular the lateral walls 25 and 26, to have a high quality hold, even at high temperatures, when the assembly 15 is heated. This is also possible because the different metals or metal alloys which the components 11 and 13 are made of have different thermal expansions. For example, the thermal expansion of the cobalt-based alloy which the second component 13 can be made of is higher than that of the titanium-based alloy which the first component 11 can be made of. This allows to increase the compressive forces between the two components 11 and 13 in the zone Z of contact between the perimeter parts 12 and 14. In this way, the inert gas, for example argon, which presses the assembly in the isostatic press 16 does not infiltrate between the components 11 and 13.

During the hot isostatic pressing process inside the isostatic press 16, advantageously, no additional equipment is necessary to create the union between the components 11 and 13, as is the case in the methods and systems known in the sector aimed at obtaining a union of components by means of diffusion bonding. This is important to avoid the contact of external material with the porous structure of the external surface 19 of the first component 11, which with pressure can lead to a slight plastic deformation or a change in the morphology of the porous structure created.

The size of the segment of interference or contact between the perimeter parts 12 and 14, therefore in particular between the lateral walls 25 and 26, can be defined as a function of the pressure of the gas applied by the HIP (Hot Isostatic Pressing).

It is of fundamental importance that the interference that is created between the two components 11 and 13 does not allow the gas to enter the interface G during the process in the isostatic press 16, since the metallurgical bond by means of diffusion bonding has to be completed between the different metals or metal alloys which the components 11 and 13 are made of.

The surface roughness of the concave seating 17 and of the cap 21 influences the quality of the bond by diffusion bonding and is preferably about 1 um Ra or less, preferably about 0.2 μm Ra or less.

The surfaces of the concave seating 17 and of the cap 21 are preferably cleaned before their assembly, so as to ensure the absence of contaminating materials of various types, such as oils used in previous working and forming processes, or other.

Thanks to the process in the isostatic press 16, the interdiffusion of atoms at the G interface takes place homogeneously, therefore the resulting bond by diffusion bonding is uniform.

In the treatment inside the isostatic press 16, it is possible to provide to keep the temperature below the “beta transus” of the titanium alloy. This allows to maintain good results in terms of mechanical properties without reducing the fatigue behavior of the final prosthetic implant 10.

After the treatment in the isostatic press 16, from which the assembly 15 exits with the components 11 and 13 joined by means of diffusion bonding, a final working step is provided.

As can be seen by comparing FIG. 3 and FIG. 4, the perimeter parts 12 and 14 that allowed the coupling by interference of the two components 11 and 13 before and during the hot isostatic pressing process are removed, together with the shank 20. A final cleaning and/or polishing step can be provided for the concave seating 24 of the cap 21 and therefore of the second component 13 and/or for the external surface 19 of the first component 11.

FIG. 5 shows under the microscope part of the interface G of contact between the components 11 and 13 once the union by diffusion bonding has been completed.

It is clear that modifications and/or additions of steps or parts may be made to the method for producing a prosthetic implant and to the prosthetic implant as described heretofore, without thereby departing from the field and scope of the present invention, as defined by the claims.

It is also clear that, although the present invention has been described with reference to some specific examples, a person of skill in the art will be able to achieve other equivalent forms of method for producing a prosthetic implant and of prosthetic implant thus achieved, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. A method for producing a prosthetic implant, the method comprising:
providing a first component made of a first metal or metal alloy and equipped with a first perimeter part having a first lateral wall;
providing a second component made of a second metal or metal alloy and equipped with a second perimeter part having a second lateral wall;
creating an assembly formed by said first component and said second component, via a coupling interference between said first perimeter part and said second perimeter part, wherein said coupling interference occurs in a zone of contact between the first lateral wall and the second lateral wall;
inserting said assembly into an isostatic press for a given period of time until diffusion bonding is obtained between said first component and said second component by means of hot isostatic pressing.

2. The method of claim 1, wherein said first component comprises a concave seating with a hemispherical or at least partly hemispherical shape, and said second component comprises a cap with a hemispherical or at least partly hemispherical shape substantially mating with the shape of said concave seating.

3. The method of claim 2, wherein between said concave seating and said cap an interface is created with a substantially hemispherical or at least partly hemispherical shape along which said diffusion bonding is made.

4. The method of claim 3, wherein said interface defines an absence of direct tight contact between said first component and said second component

5. The method of claim 1, wherein said coupling by interference occurs in a zone of contact between the lateral walls of said perimeter parts

6. The method of claim 5, wherein said contact zone (Z) is a substantially annular zone of interference between said first perimeter part (12) and said second perimeter part (14).

7. The method of claim 5, wherein said contact zone creates a region of closed porosity, defined as porosity not accessible to inert gas provided in said isostatic press between said first component and said second component.

8. The method of claim 7, wherein said coupling by interference between said first perimeter part and said second perimeter part is suitable to create said closed porosity between said first component and said second component (13).

9. The method of claim 8, wherein said first metal or metal alloy and said second metal or metal alloy have different thermal expansions.

10. The method of claim 9, wherein said coupling by interference exploits differences in thermal expansion between the two different metal materials of said first component and of said second component which, during a first thermal cycle phase, allows to increase the forces in said zone of contact between said first perimeter part and said second perimeter part, and to have a high hold, when said assembly is heated.

11. The method of claim from 7, wherein a size of the segment in which said coupling by interference occurs between said first perimeter part and said second perimeter part is defined as a function of the pressure of inert gas applied by said isostatic press,

12. The method of claim 7, wherein said interference that is created between said first perimeter part and said second perimeter part does not allow said inert gas to enter said interface during the process in said isostatic press allowing to obtain the metallurgical bond by means of diffusion bonding between different metals or metal alloys with which said first component and said second component are made.

13. The method of claim 1, wherein said first component is made using a titanium-based alloy.

14. The method of claim 1, wherein the external surface of said first component has a porous or trabecular structure.

15. The method of claim 1, wherein said second component is made using a cobalt-based alloy.

16. The method of claim 1, wherein said first perimeter part and said second perimeter part are cylindrical.

17. The method of claim 1, wherein said first perimeter part and said second perimeter part are truncated conical.

18. The method of claim 1, wherein said first perimeter part provides a seating in which said second perimeter part conformed substantially as a ring, is inserted.

19. The method of claim 2, wherein the surface roughness of said concave seating and of said cap is about 1 μm Ra or less.

20. The method of claim 1, wherein it provides a final finishing working step that follows the exit of said assembly from said isostatic press

21. The method of claim 1, wherein it provides a sintering step prior to said hot isostatic pressing step.

22. (canceled)
